(19) United States
(12) Patent Application Publication
(10) Pub. No.: US 2004/0058898 A1
(43) Pub. Date: Mar. 25, 2004

(54) Pharmaceutical composition for alleviating the violence of a mammal comprising nortestosterone derivatives

(76) Inventor: Chang-Eun Jung, Choongchunamdo (KR)

Correspondence Address: Richard P. Gilly, Wolf, Block, Schorr and Solis-Cohen LLP, 22nd Floor, 1650 Arch Street, Philadelphia, PA, 19103, US

(21) Appl. No.: 10/368,111

(22) Filed: Feb. 18, 2003

(30) Foreign Application Priority Data
Sep. 25, 2002 (KR) 10-2002-0058208

(51) Int. Cl. A61K 31/57

(52) U.S. Cl. 514/178

(57) ABSTRACT

The present invention relates to a pharmaceutical composition for alleviating the violence, sublimating the nature and stimulating the appetite of a mammal, including an ostrich, a cat, a dog, a goat, a horse, a lion, a tiger, a leopard, an elephant and pet animals, which violently behaves, cries and does not take feed, comprising an effective amount of one or more metabolic nortestosterone derivative selected from the group consisting of 17β-hydroxy-4-estren-3-one (19-nortestosterone) and ester derivatives of 17β-hydroxy-4-estren-3-one (19-nortestosterone) which is administered to said mammal in need of treatment for such conditions.

### TECHNICAL FIELD

The present invention relates to a method for alleviating the violence, sublimating the nature and stimulating the appetite of a mammal, including an ostrich, a cat, a dog, goat, a horse, a lion, a tiger, a leopard, an elephant and pet animals, which violently behaves, cries and does not take feed, comprising administrating one or more metabolic nortestosterone derivative selected from the group consisting of 17β-hydroxy-4-estren-3-one (19-nortestosterone) and ester derivatives of 17β-hydroxy-4-estren-3-one (19-nortestosterone) to said mammal in need thereof. More particularly, the present invention relates to a pharmaceutical composition for alleviating the violence, sublimating the nature and stimulating the appetite of a mammal, including an ostrich, a cat, a dog, a goat, a horse, a lion, a tiger, a leopard, an elephant and pet animals, which violently behaves, cries and does not take feed, comprising one or more metabolic nortestosterone derivative selected from the group consisting of 17β-hydroxy-4-estren-3-one (19-nortestosterone) and ester derivatives of 17β-hydroxy-4-estren-3-one (19-nortestosterone) which is administered to said mammal in need of treatment for such conditions.

### BACKGROUND ART

Some of mammals bred in Korea, including an ostrich, a cat, a dog, a goat, a horse, a lion, a tiger, a leopard, an elephant and pet animals sometimes behaves violently, cries and fasts without taking feed. Such violent behaviors, crying and feed fasting of the mammals are deeply associated with growth stage, photoperiodism and hormones.

Particularly, the mammals become violently at estrus. When the mammals at estrus were kept together in one cage, they butt, bit off, claw and outrageously fight each other, which may lead to death of animals. Also, most of the mammals at estrus not only become outrageous but also hardly take feed and some of them may be ultimately killed.

Since the ostriches, cats, dogs, goats, horses, lions, tigers, leopards, elephants and pet animals bred in Korea are mostly imported from foreign countries, breeders of these animals keenly demand research and studies for prevention of violence of the animals.

It has been known that a hormone secreted from testicles, called testosterone is involved in growth of muscle tissue and bone of animals and thus, research and studies to promote or induce growth of animals by administering testosterone to ostriches, cats, dogs, goats, horses, lions, tigers, leopards, elephants and pet animals have been conducted. However, there are problems of expensive cost for treatment of the hormone and side effects induced by treatment of testosterone.

### DISCLOSURE OF INVENTION

Therefore, the present inventors extensively studied to alleviate the violent nature and stimulating the appetite in ostriches, cats, dogs, goat, horses, lions, tigers, leopards, elephants and pet animals at estrus, thereby sublimating the violent nature and have completed the present invention based on the result of our study. It is, thus, an object of the present invention to provide a method for alleviating the violence, sublimating the nature and stimulating the appetite of mammals including ostriches, cats, dogs, goat, horses, lions, tigers, leopards, elephants and pet animals comprising administering one or more metabolic nortestosterone derivative selected from the group consisting of 17β-hydroxy-4-estren-3-one (19-nortestosterone) and ester derivatives of 17β-hydroxy-4-estren-3-one (19-nortestosterone) to said mammals in need thereof.

To achieve the above object, there is provided a method for alleviating the violence, sublimating the nature and stimulating the appetite of a mammal, including an ostrich, a cat, a dog, goat, a horse, a lion, a tiger, a leopard, an elephant and pet animals comprising orally or parenterally administrating one or more metabolic nortestosterone derivative selected from the group consisting of 17β-hydroxy-4-estren-3-one (19-nortestosterone) and ester derivatives of 17β-hydroxy-4-estren-3-one (19-nortestosterone) to said mammal in need thereof.

### BEST MODE FOR CARRYING OUT THE INVENTION

The above and other objects, features and other advantages of the present invention will be more apparent to a person skilled in the art after reading the following detailed description of the invention. Now, construction and operation of the present invention will be explained in detail.

The present invention is directed to a method for alleviating the violence, sublimating the nature and stimulating the appetite of a mammal, including an ostrich, a cat, a dog, goat, a horse, a lion, a tiger, a leopard, an elephant and pet animals, which violently behaves, cries and does not take feed, comprising orally or parenterally administrating one or more metabolic nortestosterone derivative selected from the group consisting of 17β-hydroxy-4-estren-3-one (19-nortestosterone) and ester derivatives of 17β-hydroxy-4-estren-3-one (19-nortestosterone) to said mammal in need of treatment for such conditions.

In another aspect, the present invention is also directed to a pharmaceutical composition for alleviating the violence, sublimating the nature and stimulating the appetite of a mammal, including an ostrich, a cat, a dog, a goat, a horse, a lion, a tiger, a leopard, an elephant and pet animals, which violently behaves, cries and does not take feed, comprising one or more metabolic nortestosterone derivative selected from the group consisting of 17β-hydroxy-4-estren-3-one (19-nortestosterone) and ester derivatives of 17β-hydroxy-4-estren-3-one (19-nortestosterone) which is administered to said mammal in need of treatment for such conditions.

The amount of one or more metabolic nortestosterone derivative selected from the group consisting of 17β-hydroxy-4-estren-3-one (19-nortestosterone) and ester derivatives of 17β-hydroxy-4-estren-3-one (19-nortestosterone) which can be administered according to the present invention is 0.01 to 100 mg, preferably 0.1 to 10 mg, more preferably 0.5 to 5 mg per kg of body weight of a stag. The metabolic nortestosterone derivative selected from the group consisting of 17β-hydroxy-4-estren-3-one (19-nortestosterone) and ester derivatives of 17β-hydroxy-4-estren-3-one (19-nortestosterone) can be administered orally or by injection. The ester derivatives of 17β-hydroxy-4-estren-3-one (19-nortestosterone) which can be used in the present invention include any ester derivative produced by reacting 17β-hydroxy-4-estren-3-one (19-nortestosterone) with any carboxylic acid capable of binding to 17β-hydroxy group of 17β-hydroxy-4-estren-3-one (19-nortestosterone). Preferred examples of the ester derivatives of 17β-hydroxy-4-estren-3-one (19-nortestosterone) include 19-nortestosterone benzoate, 19-nortestosterone cyclohexanecarboxylate, 19-nortestosterone cyclohexanepropionate, 19-nortestosterone decanoate, 19-nortestosterone dodecanoate, 19-nortestosterone p-hexyloxyphenylpropionate, 19-nortestosterone phenpropionate and 19-nortestosterone propionate.

According to the present invention, the carboxylic acid capable of binding to 17β-hydroxy group of 17β-hydroxy-4-estren-3-one (19-nortestosterone) includes carboxylic acids containing an alkyl group having carbon atoms of 1 to 20, a cycloalkyl group having carbon atoms of 1 to 20, a phenyl group or a phenyl substituted by an alkyl group having carbon atoms of 1 to 20. Preferred examples of the carboxylic acid capable of binding to 17β-hydroxy group of 17β-hydroxy-4-estren-3-one (19-nortestosterone) include acetic acid, benzoic acid, cyclohexanecarboxylic acid, cyclohexanepropionic acid, decanoic acid, dodecanoic acid, p-hexyloxyphenylpropionic acid, phenpropionic acid and propionic acid.

Now, the present invention will be explained in further detail using the following experimental examples. However, the present invention is not limited thereto.

### EXPERIMENTAL EXAMPLE 1

Alleviation of the Violence and Stimulation of the Appetite in Ostrich by Administration of 17β-decanoate-4-estren-3-one

Four male ostriches of body weight 100 kg which violently behaved, cried and did not take feed were intramuscularly injected with 100 mg of 17β-decanoate-4-estren-3-one (corresponding to an effective amount of 1 mg per kg of body weight of an ostrich) using 2 mL of ANABOLIN, a commercially available injection solution containing 50 mg of 17β-hydroxy-4-estren-3-one per 1 mL). After administration, their behaviors and appetites were observed. The results are shown in Table 1.

TABLE 1
Alleviation of the violence and stimulation of the appetite in male ostrich by
administration of 17β-decanoate-4-estren-3-one

| | Body | | | Violence and appetite | |
|---|---|---|---|---|---|
| | weight | Injection | Amount | Before | After |
| Race | (kg) | date | (mg) | administration | administration |
| Ostrich | 100 | 2 Oct. 2001 | 100 | Violence | Alleviated violence and |
| 1 | | | | | stimulated appetite |
| Ostrich | 100 | 2 Oct. 2001 | 100 | Violence | Alleviated violence and |
| 2 | | | | | stimulated appetite |
| Ostrich | 100 | 2 Oct. 2001 | 100 | Violence | Alleviated violence and |
| 3 | | | | | stimulated appetite |
| Ostrich | 100 | 2 Oct. 2001 | 100 | Violence | Alleviated violence and |
| 4 | | | | | stimulated appetite |

As seen from Table 1, it was shown that after intramuscular injection of 17β-decanoate-4-estren-3-one, the four male ostriches of body weight 100 kg which had violently behaved, cried and not taken feed had the violence of estrus alleviated and the appetite stimulated, thereby an increased feed intake.

### EXPERIMENTAL EXAMPLE 2

Alleviation of the Violence and Stimulation of the Appetite in Male Ostrich According to Administered Amount of 17β-decanoate-4-estren-3-one

Eight male ostriches of body weight 100 kg which violently behaved, cried and did not take feed were intramuscularly injected with 10 mg, 20 mg, 50 mg, 100 mg, 150 mg, 200 mg, 500 mg and 1000 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of an ostrich) using 0.2 mL, 0.4 mL, 1 mL, 2 mL, 3 mL, 4 mL, 10 mL and 20 mL of ANABOLIN, a commercially available injection solution containing 50 mg of 17β-hydroxy-4-estren-3-one per 1 mL). After administration, their behaviors and appetites were observed. The results are shown in Table 2.

TABLE 2
Alleviation of the violence and stimulation of the appetite in male
ostrich according to administered amount of
17β-decanoate-4-estren-3-one

| | Body | | Violence and appetite | |
|---|---|---|---|---|
| | weight | Amount | Before | After |
| Race | (kg) | (mg) | administration | administration |
| Ostrich 5 | 100 | 10 | Violence | Alleviated violence and |
| | | | | stimulated appetite |
| Ostrich 6 | 100 | 20 | Violence | Alleviated violence and |
| | | | | stimulated appetite |
| Ostrich 7 | 100 | 50 | Violence | Alleviated violence and |
| | | | | stimulated appetite |
| Ostrich 8 | 100 | 100 | Violence | Alleviated violence and |
| | | | | stimulated appetite |
| Ostrich 9 | 100 | 150 | Violence | Alleviated violence and |
| | | | | stimulated appetite |
| Ostrich 10 | 100 | 200 | Violence | Alleviated violence and |
| | | | | stimulated appetite |
| Ostrich 11 | 100 | 500 | Violence | Alleviated violence and |
| | | | | stimulated appetite |
| Ostrich 12 | 100 | 1000 | Violence | Alleviated violence and |
| | | | | stimulated appetite |

As seen from Table 2, it was shown that after intramuscular injection of 17β-hydroxy-4-estren-3-one, the male ostriches of body weight 100 kg which had violently behaved, cried and not taken feed had the violence of estrus alleviated and the appetite stimulated, thereby an increased feed intake.

### EXPERIMENTAL EXAMPLE 3

Alleviation of the Violence and Stimulation of the Appetite in Male Ostrich by Administration of 17β-cyclohexylpropinoate-4-estren-3-one

The experiment were performed by following the same procedures with Experimental Example 1, except that 17β-cyclohexylpropinoate-4-estren-3-one was used instead of 17β-decanoate-4-estren-3-one. The results of the experiment using 17β-cyclohexylpropinoate-4-estren-3-one were the same with the result of the experiment using 17β-decanoate-4-estren-3-one in Experimental Example 1.

### EXPERIMENTAL EXAMPLE 4

Alleviation of the Violence and Stimulation of the Appetite in Male Ostrich According to Administered Amount of 17β-cyclohexylpropinoate-4-estren-3-one

The experiment were performed by following the same procedures with Experimental Example 1, except that 17β-cyclohexylpropinoate-4-estren-3-one was used instead of 17β-decanoate-4-estren-3-one. The results of the experiment using 17β-cyclohexylpropinoate-4-estren-3-one were the same with the result of the experiment using 17β-decanoate-4-estren-3-one in Experimental Example 1.

### EXPERIMENTAL EXAMPLE 5

Alleviation of the Violence and Stimulation of the Appetite in Male Ostrich by Administration of Mixture of 17β-decanoate-4-estren-3-one and 17β-cyclohexylpropinoate-4-estren-3-one

The experiment were performed by following the same procedures with Experimental Example 1, except that a mixture of 17β-decanoate-4-estren-3-one and 17β-cyclohexylpropinoate-4-estren-3-one was used instead of 17β-decanoate-4-estren-3-one. The results of the experiment using the mixture of 17β-decanoate-4-estren-3-one and 17β-cyclohexylpropinoate-4-estren-3-one were the same with the result of the experiment using 17β-decanoate-4-estren-3-one in Experimental Example 1.

### EXPERIMENTAL EXAMPLE 6

Alleviation of the Violence and Stimulation of the Appetite in Male Ostrich by Administration of 17β-hydroxy-4-estren-3-one

The experiment were performed by following the same procedures with Experimental Example 1, except that 17β-hydroxy-4-estren-3-one was used instead of 17β-decanoate-4-estren-3-one. The results of the experiment using 17β-hydroxy-4-estren-3-one were the same with the result of the experiment using 17β-decanoate-4-estren-3-one in Experimental Example 1.

### EXPERIMENTAL EXAMPLE 7

Alleviation of the Violence and Stimulation of the Appetite in Male Ostrich According to Administered Amount of 17β-hydroxy-4-estren-3-one

The experiment were performed by following the same procedures with Experimental Example 2, except that 17β-hydroxy-4-estren-3-one was used instead of 17β-decanoate-4-estren-3-one. The results of the experiment using 17β-hydroxy-4-estren-3-one were the same with the result of the experiment using 17β-decanoate-4-estren-3-one in Experimental Example 2.

### EXPERIMENTAL EXAMPLE 8

Alleviation of the Violence and Stimulation of the Appetite in Male Ostrich by Administration of 17β-benzoate-4-estren-3-one

The experiment were performed by following the same procedures with Experimental Example 1, except that 17β-benzoate-4-estren-3-one was used instead of 17β-decanoate-4-estren-3-one. The results of the experiment using 17β-benzoate-4-estren-3-one were the same with the result of the experiment using 17β-decanoate-4-estren-3-one in Experimental Example 1.

### EXPERIMENTAL EXAMPLE 9

Alleviation of the Violence and Stimulation of the Appetite in Male Ostrich According to Administered Amount of 17β-benzoate-4-estren-3-one

The experiment were performed by following the same procedures with Experimental Example 2, except that 17β-benzoate-4-estren-3-one was used instead of 17β-decanoate-4-estren-3-one. The results of the experiment using 17β-benzoate-4-estren-3-one were the same with the result of the experiment using 17β-decanoate-4-estren-3-one in Experimental Example 2.

### EXPERIMENTAL EXAMPLE 10

Alleviation of the Violence and Stimulation of the Appetite in Male Ostrich by Administration of 17β-cyclohexanecarboxylate-4-estren-3-one

The experiment were performed by following the same procedures with Experimental Example 1, except that 17β-cyclohexanecarboxylate-4-estren-3-one was used instead of 17β-decanoate-4-estren-3-one. The results of the experiment using 17β-cyclohexanecarboxylate-4-estren-3-one were the same with the result of the experiment using 17β-decanoate-4-estren-3-one in Experimental Example 1.

### EXPERIMENTAL EXAMPLE 11

Alleviation of the Violence and Stimulation of the Appetite in Male Ostrich According to Administered Amount of 17β-cyclohexanecarboxylate-4-estren-3-one

The experiment were performed by following the same procedures with Experimental Example 2, except that 17β-cyclohexanecarboxylate-4-estren-3-one was used instead of 17β-decanoate-4-estren-3-one. The results of the experiment using 17β-cyclohexanecarboxylate-4-estren-3-one were the same with the result of the experiment using 17β-decanoate-4-estren-3-one in Experimental Example 2.

### EXPERIMENTAL EXAMPLE 12

Alleviation of the Violence and Stimulation of the Appetite in Male Ostrich by Administration of 17β-dodecanoate-4-estren-3-one

The experiment were performed by following the same procedures with Experimental Example 1, except that 17β-dodecanoate-4-estren-3-one was used instead of 17β-decanoate-4-estren-3-one. The results of the experiment using 17β-dodecanoate-4-estren-3-one were the same with the result of the experiment using 17β-decanoate-4-estren-3-one in Experimental Example 1.

### EXPERIMENTAL EXAMPLE 13

Alleviation of the Violence and Stimulation of the Appetite in Male Ostrich According to Administered Amount of 17β-dodecanoate-4-estren-3-one

The experiment were performed by following the same procedures with Experimental Example 2, except that 17β-dodecanoate-4-estren-3-one was used instead of 17β-decanoate-4-estren-3-one. The results of the experiment using 17β-dodecanoate-4-estren-3-one were the same with the result of the experiment using 17β-decanoate-4-estren-3-one in Experimental Example 2.

### EXPERIMENTAL EXAMPLE 14

Alleviation of the Violence and Stimulation of the Appetite in Male Ostrich by Administration of 17β-p-hexyloxyphenylpropionate-4-estren-3-one

The experiment were performed by following the same procedures with Experimental Example 1, except that 17β-p-hexyloxyphenylpropionate-4-estren-3-one was used instead of 17β-decanoate-4-estren-3-one. The results of the experiment using 17β-p-hexyloxyphenylpropionate-4-estren-3-one were the same with the result of the experiment using 17β-decanoate-4-estren-3-one in Experimental Example 1.

### EXPERIMENTAL EXAMPLE 15

Alleviation of the Violence and Stimulation of the Appetite in Male Ostrich According to Administered Amount of 17β-p-hexyloxyphenylpropionate-4-estren-3-one

The experiment were performed by following the same procedures with Experimental Example 2, except that 17β-p-hexyloxyphenylpropionate-4-estren-3-one was used instead of 17β-decanoate-4-estren-3-one. The results of the experiment using 17β-p-hexyloxyphenylpropionate-4-estren-3-one were the same with the result of the experiment using 17β-decanoate-4-estren-3-one in Experimental Example 2.

### EXPERIMENTAL EXAMPLE 16

Alleviation of the Violence and Stimulation of the Appetite in Male Ostrich by Administration of 17β-phenpropionate-4-estren-3-one

The experiment were performed by following the same procedures with Experimental Example 1, except that 17β-phenpropionate-4-estren-3-one was used instead of 17β-decanoate-4-estren-3-one. The results of the experiment using 17β-phenpropionate-4-estren-3-one were the same with the result of the experiment using 17β-decanoate-4-estren-3-one in Experimental Example 1.

### EXPERIMENTAL EXAMPLE 17

Alleviation of the Violence and Stimulation of the Appetite in Male Ostrich According to Administered Amount of 17β-phenpropionate-4-estren-3-one

The experiment were performed by following the same procedures with Experimental Example 2, except that 17β-phenpropionate-4-estren-3-one was used instead of 17β-decanoate-4-estren-3-one. The results of the experiment using 17β-phenpropionate-4-estren-3-one were the same with the result of the experiment using 17β-decanoate-4-estren-3-one in Experimental Example 2.

### EXPERIMENTAL EXAMPLE 18

Alleviation of the Violence and Stimulation of the Appetite in Male Ostrich by Administration of 17β-propionate-4-estren-3-one

The experiment were performed by following the same procedures with Experimental Example 1, except that 17β-propionate-4-estren-3-one was used instead of 17β-decanoate-4-estren-3-one. The results of the experiment using 17β-propionate-4-estren-3-one were the same with the result of the experiment using 17β-decanoate-4-estren-3-one in Experimental Example 1.

### EXPERIMENTAL EXAMPLE 19

Alleviation of the Violence and Stimulation of the Appetite in Male Ostrich According to Administered Amount of 17β-propionate-4-estren-3-one

The experiment were performed by following the same procedures with Experimental Example 2, except that 17β-propionate-4-estren-3-one was used instead of 17β-decanoate-4-estren-3-one. The results of the experiment using 17β-propionate-4-estren-3-one were the same with the result of the experiment using 17β-decanoate-4-estren-3-one in Experimental Example 2.

### EXPERIMENTAL EXAMPLE 20

Alleviation of the Violence and Stimulation of the Appetite in Dog by Administration of 17β-decanoate-4-estren-3-one

Four male dogs of body weight 40 kg which violently behaved, cried and did not take feed were intramuscularly injected with 40 mg of 17β-decanoate-4-estren-3-one (corresponding to an effective amount of 1 mg per kg of body weight of a dog) using 0.8 mL of ANABOLIN, a commercially available injection solution containing 50 mg of 17β-hydroxy-4-estren-3-one per 1 mL). After administration, their behaviors and appetites were observed. The results are shown in Table 3.

TABLE 3
Alleviation of the violence and stimulation of the appetite in male dog by administration
of 17β-decanoate-4-estren-3-one

| | Body | | | Violence and appetite | |
|---|---|---|---|---|---|
| | weight | Injection | Amount | Before | After |
| Race | (kg) | date | (mg) | administration | administration |
| Dog 1 | 40 | 2 Oct. 2001 | 40 | Violence | Alleviated violence and |
| | | | | | stimulated appetite |
| Dog 2 | 40 | 2 Oct. 2001 | 40 | Violence | Alleviated violence and |
| | | | | | stimulated appetite |
| Dog 3 | 40 | 2 Oct. 2001 | 40 | Violence | Alleviated violence and |
| | | | | | stimulated appetite |
| Dog 4 | 40 | 2 Oct. 2001 | 40 | Violence | Alleviated violence and |
| | | | | | stimulated appetite |

As seen from Table 3, it was shown that after intramuscular injection of 17β-decanoate-4-estren-3-one, the four male dogs of body weight 40 kg which had violently behaved, cried and not taken feed had the violence of estrus alleviated and the appetite stimulated, thereby an increased feed intake.

### EXPERIMENTAL EXAMPLE 21

Alleviation of the Violence and Stimulation of the Appetite in Male Dog According to Administered Amount of 17β-decanoate-4-estren-3-one

Eight male dogs of body weight 40 kg which violently behaved, cried and did not take feed were intramuscularly injected with 4 mg, 8 mg, 20 mg, 40 mg, 60 mg, 80 mg, 200 mg and 400 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of a dog) using 0.08 mL, 0.16 mL, 0.4 mL, 0.8 mL, 1.2 mL, 1.6 mL, 4 mL and 8 mL of ANABOLIN, a commercially available injection solution containing 50 mg of 17β-hydroxy-4-estren-3-one per 1 mL). After administration, their behaviors and appetites were observed. The results are shown in Table 4.

TABLE 4
Alleviation of the violence and stimulation of the appetite in male dog
according to administered amount of 17β-decanoate-4-estren-3-one

| | Body | | Violence and appetite | |
|---|---|---|---|---|
| | weight | Amount | Before | After |
| Race | (kg) | (mg) | administration | administration |
| Dog 5 | 40 | 4 | Violence | Alleviated violence and |
| | | | | stimulated appetite |
| Dog 6 | 40 | 8 | Violence | Alleviated violence and |
| | | | | stimulated appetite |
| Dog 7 | 40 | 20 | Violence | Alleviated violence and |
| | | | | stimulated appetite |
| Dog 8 | 40 | 40 | Violence | Alleviated violence and |
| | | | | stimulated appetite |
| Dog 9 | 40 | 60 | Violence | Alleviated violence and |
| | | | | stimulated appetite |
| Dog 10 | 40 | 80 | Violence | Alleviated violence and |
| | | | | stimulated appetite |
| Dog 11 | 40 | 200 | Violence | Alleviated violence and |
| | | | | stimulated appetite |
| Dog 12 | 40 | 400 | Violence | Alleviated violence and |
| | | | | stimulated appetite |

As seen from Table 2, it was shown that after intramuscular injection of 17β-hydroxy-4-estren-3-one, the male dogs of body weight 40 kg which had violently behaved, cried and not taken feed had the violence of estrus alleviated and the appetite stimulated, thereby an increased feed intake.

### EXPERIMENTAL EXAMPLE 22

Alleviation of the Violence and Stimulation of the Appetite in Cat by Administration of 17β-decanoate-4-estren-3-one

Four male cats of body weight 1 kg which violently behaved, cried and did not take feed were intramuscularly injected with 100 mg of 17β-decanoate-4-estren-3-one (corresponding to an effective amount of 1 mg per kg weight of a cat) using 0.02 mL of ANABOLIN, a commercially available injection solution containing 50 mg of 17β-hydroxy-4-estren-3-one per 1 mL). After administration, their behaviors and appetites were observed. The results are shown in Table 5.

TABLE 5
Alleviation of the violence and stimulation of the appetite in male cat by administration
of 17β-decanoate-4-estren-3-one

| | Body | | | Violence and appetite | |
|---|---|---|---|---|---|
| | weight | Injection | Amount | Before | After |
| Race | (kg) | date | (mg) | administration | administration |
| Cat 1 | 1 | 2 Oct. 2001 | 1 | Violence | Alleviated violence and |
| | | | | | stimulated appetite |
| Cat 2 | 1 | 2 Oct. 2001 | 1 | Violence | Alleviated violence and |
| | | | | | stimulated appetite |
| Cat 3 | 1 | 2 Oct. 2001 | 1 | Violence | Alleviated violence and |
| | | | | | stimulated appetite |
| Cat 4 | 1 | 2 Oct. 2001 | 1 | Violence | Alleviated violence and |
| | | | | | stimulated appetite |

As seen from Table 5, it was shown that after intramuscular injection of 17β-decanoate-4-estren-3-one, the four male cats of body weight 1 kg which had violently behaved, cried and not taken feed had the violence of estrus alleviated and the appetite stimulated, thereby an increased feed intake.

### EXPERIMENTAL EXAMPLE 23

Alleviation of the Violence and Stimulation of the Appetite in Male Cat According to Administered Amount of 17β-decanoate-4-estren-3-one

Eight male cats of body weight 1 kg which violently behaved, cried and did not take feed were intramuscularly injected with 0.1 mg, 0.2 mg, 0.5 mg, 1.0 mg, 1.5 mg, 2.0 mg, 5.0 mg and 10 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of a cat) using 0.002 mL, 0.004 mL, 0.01 mL, 0.02 mL, 0.03 mL, 0.04 mL, 0.1 mL and 0.2 mL of ANABOLIN, a commercially available injection solution containing 50 mg of 17β-hydroxy-4-estren-3-one per 1 mL). After administration, their behaviors and appetites were observed. The results are shown in Table 6.

TABLE 6
Alleviation of the violence and stimulation of the appetite in male cat
according to administered amount of 17β-decanoate-4-estren-3-one

| | Body | | Violence and appetite | |
|---|---|---|---|---|
| | weight | Amount | Before | After |
| Race | (kg) | (mg) | administration | administration |
| Cat 5 | 1 | 0.1 | Violence | Alleviated violence and |
| | | | | stimulated appetite |
| Cat 6 | 1 | 0.2 | Violence | Alleviated violence and |
| | | | | stimulated appetite |
| Cat 7 | 1 | 0.5 | Violence | Alleviated violence and |
| | | | | stimulated appetite |
| Cat 8 | 1 | 1 | Violence | Alleviated violence and |
| | | | | stimulated appetite |
| Cat 9 | 1 | 1.5 | Violence | Alleviated violence and |
| | | | | stimulated appetite |
| Cat 10 | 1 | 2 | Violence | Alleviated violence and |
| | | | | stimulated appetite |
| Cat 11 | 1 | 5 | Violence | Alleviated violence and |
| | | | | stimulated appetite |
| Cat 12 | 1 | 10 | Violence | Alleviated violence and |
| | | | | stimulated appetite |

As seen from Table 6, it was shown that after intramuscular injection of 17β-hydroxy-4-estren-3-one, the male cats of body weight 1 kg which had violently behaved, cried and not taken feed had the violence of estrus alleviated and the appetite stimulated, thereby an increased feed intake.

### EXPERIMENTAL EXAMPLE 24

Alleviation of the Violence and Stimulation of the Appetite in Horse by Administration of 17β-decanoate-4-estren-3-one

Four male horses of body weight 200 kg which violently behaved, cried and did not take feed were intramuscularly injected with 200 mg of 17β-decanoate-4-estren-3-one (corresponding to an effective amount of 1 mg per kg weight of a horse) using 4 mL of ANABOLIN, a commercially available injection solution containing 50 mg of 17β-hydroxy-4-estren-3-one per 1 mL). After administration, their behaviors and appetites were observed. The results are shown in Table 7.

TABLE 7
Alleviation of the violence and stimulation of the appetite in male horse by
administration of 17β-decanoate-4-estren-3-one

| | Body | | | Violence and appetite | |
|---|---|---|---|---|---|
| | weight | Injection | Amount | Before | After |
| Race | (kg) | date | (mg) | administration | administration |
| Horse | 200 | 2 Oct. 2001 | 200 | Violence | Alleviated violence and |
| 1 | | | | | stimulated appetite |
| Horse | 200 | 2 Oct. 2001 | 200 | Violence | Alleviated violence and |
| 2 | | | | | stimulated appetite |
| Horse | 200 | 2 Oct. 2001 | 200 | Violence | Alleviated violence and |
| 3 | | | | | stimulated appetite |
| Horse | 200 | 2 Oct. 2001 | 200 | Violence | Alleviated violence and |
| 4 | | | | | stimulated appetite |

As seen from Table 7, it was shown that after intramuscular injection of 17β-decanoate-4-estren-3-one, the four male horses of body weight 200 kg which had violently behaved, cried and not taken feed had the violence of estrus alleviated and the appetite stimulated, thereby an increased feed intake.

### EXPERIMENTAL EXAMPLE 25

Alleviation of the Violence and Stimulation of the Appetite in Male Horse According to Administered Amount of 17β-decanoate-4-estren-3-one

Eight male horses of body weight 200 kg which violently behaved, cried and did not take feed were intramuscularly injected with 20 mg, 40 mg, 100 mg, 200 mg, 300 mg, 400 mg, 1000 mg and 2000 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of a horse) using 0.4 mL, 0.8 mL, 2 mL, 4 mL, 6 mL, 8 mL, 20 mL and 40 mL of ANABOLIN, a commercially available injection solution containing 50 mg of 17β-hydroxy-4-estren-3-one per 1 mL). After administration, their behaviors and appetites were observed. The results are shown in Table 8.

TABLE 8
Alleviation of the violence and stimulation of the appetite in
male horse according to administered amount
of 17β-decanoate-4-estren-3-one

| | Body | | Violence and appetite | |
|---|---|---|---|---|
| | weight | Amount | Before | After |
| Race | (kg) | (mg) | administration | administration |
| Horse 5 | 200 | 20 | Violence | Alleviated violence and |
| | | | | stimulated appetite |
| Horse 6 | 200 | 40 | Violence | Alleviated violence and |
| | | | | stimulated appetite |
| Horse 7 | 200 | 100 | Violence | Alleviated violence and |
| | | | | stimulated appetite |
| Horse 8 | 200 | 200 | Violence | Alleviated violence and |
| | | | | stimulated appetite |
| Horse 9 | 200 | 300 | Violence | Alleviated violence and |
| | | | | stimulated appetite |
| Horse 10 | 200 | 400 | Violence | Alleviated violence and |
| | | | | stimulated appetite |
| Horse 11 | 200 | 1000 | Violence | Alleviated violence and |
| | | | | stimulated appetite |
| Horse 12 | 200 | 2000 | Violence | Alleviated violence and |
| | | | | stimulated appetite |

As seen from Table 8, it was shown that after intramuscular injection of 17β-hydroxy-4-estren-3-one, the male horses of body weight 200 kg which had violently behaved, cried and not taken feed had the violence of estrus alleviated and the appetite stimulated, thereby an increased feed intake.

### EXPERIMENTAL EXAMPLE 26

Alleviation of the Violence and Stimulation of the Appetite in Goat by Administration of 17β-decanoate-4-estren-3-one

Four male goats of body weight 40 kg which violently behaved, cried and did not take feed were intramuscularly injected with 40 mg of 17β-decanoate-4-estren-3-one (corresponding to an effective amount of 1 mg per kg weight of a goat) using 0.8 mL of ANABOLIN, a commercially available injection solution containing 50 mg of 17β-hydroxy-4-estren-3-one per 1 mL). After administration, their behaviors and appetites were observed. The results are shown in Table 9.

TABLE 9
Alleviation of the violence and stimulation of the appetite in male goat by
administration of 17β-decanoate-4-estren-3-one

| | Body | | | Violence and appetite | |
|---|---|---|---|---|---|
| | weight | Injection | Amount | Before | After |
| Race | (kg) | date | (mg) | administration | administration |
| Goat | 40 | 2 Oct. 2001 | 40 | Violence | Alleviated violence and |
| 1 | | | | | stimulated appetite |
| Goat | 40 | 2 Oct. 2001 | 40 | Violence | Alleviated violence and |
| 2 | | | | | stimulated appetite |
| Goat | 40 | 2 Oct. 2001 | 40 | Violence | Alleviated violence and |
| 3 | | | | | stimulated appetite |
| Goat | 40 | 2 Oct. 2001 | 40 | Violence | Alleviated violence and |
| 4 | | | | | stimulated appetite |

As seen from Table 9, it was shown that after intramuscular injection of 17-decanoate-4-estren-3-one, the four male goats of body weight 40 kg which had violently behaved, cried and not taken feed had the violence of estrus alleviated and the appetite stimulated, thereby an increased feed intake.

### EXPERIMENTAL EXAMPLE 27

Alleviation of the Violence and Stimulation of the Appetite in Male Goat According to Administered Amount of 17β-decanoate-4-estren-3-one

Eight male goats of body weight 40 kg which violently behaved, cried and did not take feed were intramuscularly injected with 4 mg, 8 mg, 20 mg, 40 mg, 60 mg, 80 mg, 200 mg and 400 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of a goat) using 0.08 mL, 0.15 mL, 0.4 mL, 0.8 mL, 1.2 mL, 1.6 mL, 4 mL and 8 mL of ANABOLIN, a commercially available injection solution containing 50 mg of 17β-hydroxy-4-estren-3-one per 1 mL). After administration, their behaviors and appetites were observed. The results are shown in Table 10.

TABLE 10
Alleviation of the violence and stimulation of the appetite in male goat
according to administered amount of 17β-decanoate-4-estren-3-one

| | Body | | Violence and appetite | |
|---|---|---|---|---|
| | weight | Amount | Before | After |
| Race | (kg) | (mg) | administration | administration |
| Goat 5 | 40 | 4 | Violence | Alleviated violence and |
| | | | | stimulated appetite |
| Goat 6 | 40 | 8 | Violence | Alleviated violence and |
| | | | | stimulated appetite |
| Goat 7 | 40 | 20 | Violence | Alleviated violence and |
| | | | | stimulated appetite |
| Goat 8 | 40 | 40 | Violence | Alleviated violence and |
| | | | | stimulated appetite |
| Goat 9 | 40 | 60 | Violence | Alleviated violence and |
| | | | | stimulated appetite |
| Goat 10 | 40 | 80 | Violence | Alleviated violence and |
| | | | | stimulated appetite |
| Goat 11 | 40 | 200 | Violence | Alleviated violence and |
| | | | | stimulated appetite |
| Goat 12 | 40 | 400 | Violence | Alleviated violence and |
| | | | | stimulated appetite |

As seen from Table 10, it was shown that after intramuscular injection of 17β-hydroxy-4-estren-3-one, the male goats of body weight 40 kg which had violently behaved, cried and not taken feed had the violence of estrus alleviated and the appetite stimulated, thereby an increased feed intake.

### EXPERIMENTAL EXAMPLE 28

Alleviation of the Violence and Stimulation of the Appetite in Lion by Administration of 17β-decanoate-4-estren-3-one

A male lion of body weight 100 kg which violently behaved, cried and did not take feed was intramuscularly injected with 100 mg of 17β-decanoate-4-estren-3-one (corresponding to an effective amount of 1 mg per kg weight of a lion) using 2 mL of ANABOLIN, a commercially available injection solution containing 50 mg of 17β-hydroxy-4-estren-3-one per 1 mL). As a result, it was shown that after intramuscular injection of 17β-decanoate-4-estren-3-one, the male lions had the violence of estrus alleviated and the appetite stimulated, thereby an increased feed intake.

### EXPERIMENTAL EXAMPLE 29

Alleviation of the Violence and Stimulation of the Appetite in Male Lion According to Administered Amount of 17β-decanoate-4-estren-3-one

A male lion of body weight 100 kg which violently behaved, cried and did not take feed was intramuscularly injected with 10 mg, 20 mg, 50 mg, 100 mg, 150 mg, 200 mg, 500 mg and 1000 mg of 17β-decanoate-4-estren-3-one (corresponding to an effective amount of 0.1 mg, 0.2 mg, 0.5 mg, 1.0 mg, 1.5 mg, 2.0 mg, 5.0 mg and 10 mg per kg weight of a lion) using 0.2 mL, 0.4 mL, 1 mL, 2 mL, 3 mL, 4 mL, 10 mL and 20 mL of ANABOLIN, a commercially available injection solution containing 50 mg of 17β-hydroxy-4-estren-3-one per 1 mL). As a result, it was shown that after intramuscular injection of 17β-decanoate-4-estren-3-one, the male lions had the violence of estrus alleviated and the appetite stimulated, thereby an increased feed intake.

### INDUSTRIAL APPLICABILITY

As described in the above experimental examples, by administering one or more metabolic nortestosterone derivative selected from the group consisting of 17β-hydroxy-4-estren-3-one and ester derivatives of 17β-hydroxy-4-estren-3-one to a mammal, including an ostrich, a cat, a dog, a goat, a horse, a lion, a tiger, a leopard, an elephant and pet animals, which violently behaves, cries and does not take feed according to the present invention, it is possible to alleviate the violence, sublimating the nature and stimulating the appetite of said mammal. Also, there can be brought an epoch-making improvement in mammal breeding. Further, the present invention can contribute to improvement in competitiveness of the zoo and mammal breeding industries by reducing the expense taken to import these animals.

In addition, according to the present invention, it is possible to breed pet animals such as dogs and cats without castration, reducing the cost related thereto and management expense.

What is claimed is:

1. A pharmaceutical composition for alleviating the violence, sublimating the nature and stimulating the appetite of a mammal, including an ostrich, a cat, a dog, a goat, a horse, a lion, a tiger, a leopard, an elephant and pet animals, which violently behaves, cries and does not take feed, comprising an effective amount of one or more metabolic nortestosterone derivative selected from the group consisting of 17β-hydroxy-4-estren-3-one(19-nortestosterone) and ester derivatives of 17β-hydroxy-4-estren-3-one which is administered to said mammal in need of treatment for such conditions.

2. The pharmaceutical composition according to claim 1, wherein the ester derivatives of 17β-hydroxy-4-estren-3-one(19-nortestosterone) comprise 19-nortestosterone benzoate, 19-nortestosterone cyclohexanecarboxylate, 19-nortestosterone cyclohexanepropionate, 19-nortestosterone decanoate, 19-nortestosterone dodecanoate, 19-nortestosterone p-hexyloxyphenylpropionate, 19-nortestosterone phenpropionate and 19-nortestosterone propionate.

3. The pharmaceutical composition according to claim 1, wherein carboxylic acids capable of binding to 17β-hydroxy group of 17β-hydroxy-4-estren-3-one (19-nortestosterone) to produce the ester derivatives of 17β-hydroxy-4-estren-3-one comprise carboxylic acids containing an alkyl group having carbon atoms of 1 to 20, a cycloalkyl group having carbon atoms of 1 to 20, a phenyl group or a phenyl substituted by an alkyl group having carbon atoms of 1 to 20.

4. The pharmaceutical composition according to claim 3, wherein the carboxylic acid capable of binding to 17β-hydroxy group of 17β-hydroxy-4-estren-3-one comprise acetic acid, benzoic acid, cyclohexanecarboxylic acid, cyclohexanepropionic acid, decanoic acid, dodecanoic acid, p-hexyloxyphenylpropionic acid, phenpropionic acid and propionic acid.

5. The pharmaceutical composition according to claim 1, wherein the effective amount of one or more metabolic nortestosterone derivative selected from the group consisting of 17β-hydroxy-4-estren-3-one and ester derivatives of 17β-hydroxy-4-estren-3-one is 0.1 to 10 mg per kg of body weight of an mammal.

6. The pharmaceutical composition according to claim 1, wherein the composition is administered orally or by injection.
